(19) United States
(12) Patent Application Publication
(10) Pub. No.: US 2005/0214886 A1
(43) Pub. Date: Sep. 29, 2005

(54) TNF modulation

(75) Inventors: David Wallach, Rehovot (IL); Eva Pocsik, Budapest (HU); Erno Duda, Szeged (HU)

Correspondence Address: BROWDY AND NEIMARK, P.L.L.C.;624 NINTH STREET, NW, SUITE 300, WASHINGTON, DC, 20001-5303, US

(73) Assignee: YEDA RESEARCH AND DEVELOPMENT CO. LTD., Rehovot (IL)

(21) Appl. No.: 10/968,191

(22) Filed: Oct. 20, 2004

(30) Foreign Application Priority Data
Jul. 5, 1995 (IL) 114,461

(51) Int. Cl. C12Q 1/48

(52) U.S. Cl. 435/15

(57) ABSTRACT

Modulators such as proteins, peptides, antibodies or analogs, fragments or derivatives of any of them are provided which are capable of interacting with, or binding to, the intracellular domain of the membrane-bound form of TNF (26 KD TNF). These modulators are capable of regulating the expression, proteolytic processing, bioactivity or intracellular signaling of the 26 KD TNF and may thus be used for the treatment of diseases in which TNF plays a central role.

### FIELD OF THE INVENTION

The present invention is generally in the field of the regulation of the activity of the pleotropic cytokine, tumor necrosis factor (TNF). More specifically, the present invention concerns new modulators such as proteins, peptides, antibodies or analogs, fragments or derivatives of any thereof, and organic compounds which are capable of interacting with, or binding to, the intracellular domain of the membrane-bound form of TNF (26 kDa TNF). These new modulators are capable of modulating or regulating the expression, proteolytic processing, bioactivity or intracellular signaling of the 26 kDa TNF.

### BACKGROUND OF THE INVENTION AND PRIOR ART

Tumor necrosis factor (TNF) is a pleiotropic cytokine that plays a central role in the induction of inflammation. Its wide range of effects include cytotoxicity, stimulation of cell growth and induction of changes in cell differentiation patterns and various immune modulatory activities (Aggarwal and Vilcek, 1991). It is primarily produced in mononuclear phagocytes following their stimulation with bacterial components, such as lipopolysaccharide (LPS), or viruses or multicellular parasites. TNF molecules are initially produced in the form of 26 kDa β-transmembrane proteins with a signal peptide of 76 amino acid residues (Pennica et al., 1984). These transmembrane molecules may remain on the surface of the cells that produce them or are proteolytically processed, yielding soluble 17 kDa TNF molecules (Kriegler et al., 1988; Perez et al., 1990; Jue et al., 1990).

Both the cell surface and soluble forms of TNF can trigger effects characteristic of this cytokine in target cells by binding to the same two species of TNF receptors, the p55 TNF-R and the p75 TNF-R (Kriegler et al., 1988; Perez et al., 1990; Decker, et al., 1987; Peck et al., 1989; Duerksen-Hughes, et al., 1992; Nii, et al., 1993; Ratner and Clark, 1993; Lopez-Cepero et al., 1994). However, there are some differences in their mode of action resulting from the differences in their structure and physical state. The soluble form of TNF acts at a multiplicity of sites, adjacent to its formation site as well as distant from it, as is the case with other endocrine regulators, while the function of cell bound TNF is limited to the vicinity of the TNF producing cell. In addition, the mechanism of action of cell surface TNF differs from that of the soluble form in terms of the extent of influence of the individual TNF-producing cell on the nature of the effects of the cytokine. Unlike soluble TNF, and other endocrine mediators, whose mode of action is largely independent of their way of formation, cell-bound TNF molecules act in a way which dictates a direct link between TNF production and function. The location of the effector cell, the effectivity of TNF production and perhaps, also the way in which the cell presents TNF on its surface, determine the identity of the target cell and its mode of response. There also seem to be some differences in the nature of the effects induced by the two molecular forms of TNF (Peck et al., 1989; Birkland et al., 1992), suggesting that they can trigger different signaling activities. For example, recent evidence indicates that cell-surface-bound TNF stimulates the p75 TNF-R at a higher level than does soluble TNF.

As mentioned above, TNF has many effects on cells. Some of these effects are beneficial to the organism: TNF mav destroy, for example, tumor cells or virus infected cells and augment antibacterial activities of granulocytes. In this way, TNF contributes to the defence of the organism against tumors and infectious agents and contributes to the recovery from injury. Thus, TNF can be used as an antitumor agent in which application it binds to its receptors on the surface of tumor cells and thereby initiates the events leading to the death of the tumor cells. TNF can also be used as an anti-infectious agent.

However, TNF also has deleterious effects on cells. There is evidence that over-production of TNF can play a major pathogenic role in several diseases. Thus, effects of TNF, primarily on the vasculature, are now known to be a major cause for symptoms of septic shock. In fact, it has recently been shown that an inhibitor of the shedding of TNF from the cell-surface can prevent septic shock. This inhibitor acts extracellularly on the protease which cleaves the soluble TNF molecule from the cell-surface-bound TNF molecule. In some diseases, TNF may cause excessive loss of weight (cachexia) by suppressing activities of adipocytes and by causing anorexia. TNF has also been described as a mediator of the damage to tissues in rheumatic diseases, and as a major mediator of the damage observed in graft-versus-host reactions. In addition, TNF is known to be involved in the process of inflammation and in many other diseases.

It has been a long felt need to provide a way for modulating the cellular response to TNF, for example, in the above-noted pathological situations where TNF is over-expressed it is desirable to inhibit the TNF-induced cytocidal effects; while in other situations, for example, wound-healing applications, it is desirable to enhance the TNF effect.

A number of approaches have been made by the applicants (see, for example, EP 186833, EP 308378, EP 398327 and EP 412486) to regulate the deleterious effects of TNF by inhibiting the binding of TNF to its receptors using anti-TNF antibodies or by using soluble TNF receptors to compete with the binding of TNF to the cell surface-bound TNF receptors (TNF-Rs). Further, on the basis that TNF-binding to its receptors is required for the TNF-induced cellular effects, approaches by the applicants (see, for example, EP 568925) have been made to modulate the TNF effect by modulating the activity of the TNF-Rs. Briefly, EP 568925 relates to a method of modulating signal transduction and/or cleavage in TNF-Rs whereby peptides or other molecules may interact either with the receptor itself or with effector proteins interacting with the receptor, thus modulating the normal functioning of the TNF-Rs. In EPO 568925 there is described the construction and characterization of various mutant p55 TNF-Rs, having mutations in the extracellular, transmembranal, and intracellular domains of the p55 TNF-R. In this way regions within these domains were identified as being essential for the functioning of the receptor. Further, in EP 568925 there is also described a number of approaches to isolate and identify proteins, peptides or other factors which are capable of binding to the various regions in the above domains of the p55 TNF-R, which proteins, peptides and other factors may be involved in regulating or modulating the activity of the TNF-R; and there is described a number of approaches for isolating and cloning the DNA sequences encoding such proteins and peptides, for constructing expression vectors for the production of these proteins and peptides, and for the preparation of antibodies or fragments thereof which interact with the TNF-R or with the above proteins and peptides that bind various regions of the TNF-R. However, in EP 568925 no description is made of the actual proteins and peptides which bind to the intracellular domains of the TNF-Rs and which may thereby modulate the intracellular signaling process, mediated by the TNF-Rs, which ultimately results in the observed TNF-induced cellular effects.

In recent studies by the applicants (see, for example, IL 109632, IL 111125, IL 112002 and IL 112742) there has been described, amongst other aspects; a number of proteins which specifically bind to one or more of the intracellular domains of the p55 TNF-R, p75 TNF-R and the related FAS ligand receptor (FAS-R or FAS/Apo1), and which proteins or analogs, fragments or derivatives thereof may modulate the activity of these receptors by modulating the intracellular signaling process mediated by these receptors. In these co-pending applications there is also described the use of the yeast two-hybrid approach to isolate, identify and clone such intracellular domain-binding proteins, as well as a number of ways in which these intracellular domain-binding proteins may be administered or otherwise used in order to modulate the activity of the various TNF-Rs and FAS-R.

Other approaches to regulate the TNF effect on cells have also been made, by which it was sought to decrease the amount or the activity of TNF-Rs at the cell surface when it is desired to inhibit the TNF effect, or to increase the amount or the activity of TNF-Rs at the cell surface when it is desired to enhance the TNF effect. One such approach was by way of sequencing and analyzing the promoters of the p75 TNF-R and p55 TNF-R genes. This analysis yielded a number of key sequence motifs that are specific to various transcription regulatory factors, and thereby provide a way for controlling the expression of the TNF-Rs at the level of the promoters of their genes, i.e. inhibition of transcription from the promoters will result in a decrease in the number of TNF-Rs, and enhancement of transcription from the promoters will result in an increase in the number of TNF-Rs (see, for example, the co-pending applications IL 104355 and IL 109633).

Heretofore there has not been described a means for modulating the effect of TNF by modulating the amount and activity of TNF that is present at the cell surface or that is shed from the cell surface by modulation of the intracellular domain of the cell-bound form of TNF. Further, there also has not been previously described a means for modulating the effect of TNF by modulating the intracellular signaling process mediated by the intracellular domain of the cell-bound form (26 kDa form) of TNF. This intracellular signaling process mediated by the intracellular domain of TNF may be directly involved in regulating the amount of TNF that is formed in the cells. As mentioned above, cell-bound TNF molecules act in a way that dictates a direct link between TNF production and function. Further, the presence of the intracellular domain in cell-bound TNF molecules influences the way in which the cell presents TNF on its surface, which, in turn, determined the activity (function) of the TNF>

In view of the distinctive features of the mechanism of action of cell-bound TNF, some types of heretofore not described control mechanisms specifically regulating the action of these molecules are likely to exist. The intracellular domain of the membrane-associated TNF molecules may serve such a role since it is accessible to modulation by intracellular mechanisms. Although the intracellular domain of the TNF molecule has no direct involvement in receptor binding, its sequence is highly conserved among different animal species, suggesting that it has important function (reviewed in Wallach, 1986; Van Ostade et al., 1994).

It is an object of the present invention to provide a way by which signals within the TNF-producing cells can affect the function of the cell-surface TNF, and thereby provide a method for regulating TNF activity or the amount of TNF by modulating signal transduction, mediated by the intracellular domain of TNF, by way of modulating the activity of the intracellular domain of TNF or by way of modulating the activity of one or more effector proteins which interact with the intracellular domain of TNF.

Another object of the invention is to provide modulatory molecules, e.g. proteins, peptides, antibodies or organic compounds which specifically interact with the intracellular domain of TNF thereby modulating its activity, and hence which are capable of modulating the activity of TNF.

It is a further object of the invention is to provide pharmaceutical compositions comprising the above modulatory molecules for the treatment of diseases in which TNF plays a central role.

### SUMMARY OF THE INVENTION

In accordance with the present invention it has been found that the cell-surface-bound form of TNF, i.e. the 26 kDa transmembrane TNF molecules, isolated from [³²P]-labeled HeLa cells that had been transfected with a cDNA encoding a partially cleavable TNF mutant, were labeled. Phosphorylated 26 kDa TNF molecules were also isolated from LPS-stimulated human monocytic Mono Mac 6 cells. Phosphoamino acid analysis revealed that the labeled phosphate is bound to one or more serine residues in these 26 kDa TNF molecules. Since no label was found to be incorporated in the soluble 17 kDa form of TNF (which is proteolytically derived from the 26 kDa form), these findings indicate that the phosphorylated residue(s) of the membrane-associated 26 kDa TNF molecules are present in the intracellular domain of these 26 kDa TNF molecules.

Moreover, the sequence conservation of the intracellular domain of the cell-surface form of the TNF molecule in different species indicates that this domain and its phosphorylation, as found in accordance with the present invention, play important roles in TNF function. One possibility is that this domain takes part in the regulation of the proteolytic process by which the 17 kDa form of TNF is derived from the 26 kDa molecule. Another possibility is that this intracellular domain of the TNF molecule may effect TNF function as a ligand, i.e. this domain may impose conformational changes in the ligand binding of the extracellular domain of the TNF molecule, or it could dictate association with cytoskeletal elements and thus direct the translocation of the TNF molecules within the membrane towards the area of the cell surface adjacent to the target cell (i.e. another cell carrying TNF-Rs on which the cell producing membrane-bound TNF molecules acts). Yet another possibility is that the intracellular domain of TNF interacts with other intracellular molecules possessing signaling activities, and hence the activation of signaling activities within the TNF-producing cell following the interaction of the cell surface TNF with its target cell may allow a fine adjustment of the function or formation of the 26 kDa cell-surface TNF molecules.

Thus, the phosphorylation of the intracellular domain of the 26 kDa TNF molecules may be involved in the regulation of expression or proteolytic processing of cell-surface TNF, in the modulation of TNF bioactivity, or in the intracellular signaling process mediated by the cell-surface TNF molecules.

The above findings and their related functional significance represent the first disclosure of a control possibility (both in terms of biological activity and amounts) of the cell-surface form of TNF via control of the activity of the intracellular domain of this form of TNF, in particular, via control of the region in this domain which is subject to phosphorylation.

Accordingly, the present invention provides a modulator of the expression, proteolytic processing, bioactivity or intracellular signaling of the 26 kDa cell-surface-bound form of TNF (26 kDa TNF), said modulator being capable of interacting with the intracellular domain of said 26 kDa TNF or with one or more other intracellular effector proteins which interact with said intracellular domain of the 26 kDa TNF.

In particular, the present invention provides:
(a) a modulator which is selected from the group comprising: (i) naturally-derived proteins, peptides, analogs and derivatives thereof capable of interacting with said intracellular domain of 26 kDa TNF or with said other intracellular effect proteins; (ii) synthetically produced complementary peptides synthesized by using as substrate the intracellular domain or portions thereof of the 26 kDa TNF, said complementary peptides being capable of interacting with said intracellular domain of the 26 kDa TNF or with said other intracellular effector proteins; (iii) antibodies or active fragments thereof capable of interacting with said intracellular domain of the 26 kDa TNF or with said other intracellular effector proteins; and (iv) organic compounds capable of interacting with said intracellular domain of the 26 kDa TNF or with said other intracellular effector proteins, said organic compounds being derived from known compounds and selected using said intracellular domain or portions thereof of 26 kDa TNF as a substrate in a binding assay, or being synthesized using said intracellular domain or portions thereof of 26 kDa TNF as a substrate for designing and synthesizing said organic compounds;
(b) a modulator which is capable of interacting with one or more serine residues in the intracellular domain of said 26 kDa TNF which are substrates of phosphorylation, or with one or more phosphorylated serine residues in the intracellular domain of said 26 kDa TNF, or with one or more kinase enzymes which are involved in the phosphorylation of said one or more serine residues, or with one or more other intracellular effector proteins which interact with said serine or phosphorylated serine residues.

The present invention also provides a DNA sequence encoding a modulator being a protein, peptide or an analog thereof, as set forth herein above.

An embodiment of the DNA sequence of the invention is a DNA sequence encoding a naturally-derived protein or peptide selected from the group consisting of:
(a) a cDNA sequence derived from the coding region of a native 26 kDa TNF intracellular domain-binding protein or peptide;
(b) DNA sequences capable of hybridization to a sequence of (a) under moderately stringent conditions and which encode a biologically active 26 kDa TNF intracellular domain-binding protein or peptide; and
(c) DNA sequences which are degenerate as a result of the genetic code to the DNA sequenced defined in (a) and (b) and which encode a biologically active 26 kDa TNF intracellular domain-binding protein.

Furthermore, there is also provided:
(i) a protein, peptide or analogs thereof encoded by a DNA sequence of the invention, said protein, peptide and analogs being capable of binding to or interacting with the intracellular domain of the 26 kDa TNF;
(ii) a vector comprising a DNA sequence of the invention;
(iii) a vector of (ii) which is capable of being expressed in a eukaryotic or prokaryotic host cell;
(iv) transformed eukaryotic or prokaryotic host cells containing a vector of (ii) or (iii);
(v) a method for producing the protein, peptide or analogs of (i) comprising growing the transformed host cells of (iv) under conditions suitable for the expression of said protein, peptide or analogs, effecting post-translational modifications of said protein, peptide or analogs as necessary for the obtention thereof and extracting said expressed protein, peptide or analogs from the culture medium of said transformed cells or from cell extracts of said transformed cells;
(vi) antibodies or active fragments or derivatives thereof specific for the protein, peptide or analogs of (i).

The present invention also provides a method for the modulation of the expression, proteolytic processing, bioactivity or intracellular signaling of the 26 kDa TNF comprising treating cells with a modulator of the invention as noted above, or with a protein, peptide or analogs of (i) above, or with antibodies, active fragments or derivatives of (vi) above, wherein said treating of cells comprises introducing into the cells said naturally derived proteins, peptides, analogs and derivatives thereof, said complementary peptides, said antibodies, or said organic compounds in a form suitable for intracellular introduction thereof, or when said modulator is a protein, peptide or analogs thereof, said treatment of cells also comprises introducing into said cells a DNA sequence encoding said protein, peptide or analogs in the form of a suitable vector carrying said sequence, said vector being capable of effecting the insertion of said sequence into said cells in a way that said sequence is expressed in the cells.

An embodiment of the above method is a method wherein said treating of cells is by administration of said protein, peptide or analogs, and said administration is by transfection of said cells with a recombinant animal virus vector comprising the steps of:
(a) constructing a recombinant animal virus vector carrying a sequence encoding a viral surface protein (ligand) that is capable of binding to a specific cell surface receptor of the surface of said cell to be treated, and a second seequence encoding a protein, peptide or analogs of the invention, said protein, peptide or analogs when expressed in said cells being capable of modulating the expression, proteolytic processing, bioactivity or intracellular signaling of the 26 kDa TNF by interacting with the intracellular domain of said 26 kDa TNF or by interacting with another intracellular effector protein which interacts with said 26 kDa TNF intracellular domain; and
(b) infecting said cells with said vector of (a).

Another embodiment of the above method is a method wherein said treating of cells is by administration of said antibodies, active fragments or derivatives thereof of the invention, said treating being by application of a suitable composition containing said antibodies, active fragments or derivatives thereof to said cells, said composition being formulated for intracellular application.

Another method of the invention is a method for the modulation of the expression, proteolytic processing, bioactivity or intracellular signaling of the 26 kDa TNF in 26 kDa TNF-producing cells, comprising treating said cells with an oligonucleotide sequence selected from a sequence encoding an antisense sequence of at least part of the DNA sequence of the invention, said oligonucleotide sequence being capable of blocking the expression of at least one protein or peptide which interacts with the intracellular domain of the 26 kDa TNF.

An embodiment of this method is a method wherein said oligonucleotide sequence is introduced into said cells via a recombinant virus vector as noted above, wherein said second sequence of the virus encodes said oligonucleotide sequence.

Yet another method of the invention is a method for modulation of the expression, proteolytic processing, bioactivity or intracellular signaling of the 26 kDa TNF in 26 kDa TNF-producing cells, comprising applying the ribozyme procedure in which a vector encoding a ribozyme sequence capable of interacting with a cellular mRNA sequence encoding a protein or peptide of the invention, is introduced into said cells in a form that permits expression of said ribozyme sequence in said cells and wherein, when said ribozyme sequence is expressed in said cells it interacts with said cellular mRNA sequence and cleaves said mRNA sequence resulting in the inhibition of expression of said protein or peptide in said cells.

Other methods of the invention are:
(i) a method for isolating and identifying proteins, peptides, factors or receptors capable of interacting with or binding to the intracellular domain of the 26 kDa TNF comprising applying the procedure of affinity chromatography in which the intracellular domain or portions thereof of the 26 kDa TNF is attached to the affinity chromatography matrix and is brought into contact with a cell extract, and proteins, peptides, factors or receptors from the cell extract which bound to said attached 26 kDa TNF intracellular domain or portions thereof, are then eluted, isolated and analyzed;
(ii) a method for isolating and identifying proteins and peptides capable of binding to the intracellular domain of the 26 kDa TNF comprising applying the yeast two-hybrid procedure in which a sequence encoding said 26 kDa TNF intracellular domain or portions thereof is carried by one hybrid vector and a sequence from a cDNA or genomic DNA library are carried by the second hybrid vector, the vectors then being used to transform yeast host cells and the positive transformed cells being isolated, followed by extraction of said second hybrid vector to obtain a sequence encoding a protein or peptide which binds to said 26 kDa TNF intracellular domain or portions thereof;
(iii) a method for isolating and identifying a protein or peptide capable of binding to the intracellular domain of the 26 kDa TNF comprising applying the procedure of non-stringent Southern hybridization followed by PCR cloning in which a sequence or parts thereof of the invention is used as a probe to bind sequences from a cDNA or genomic DNA library having at least partial homology thereto, said bound sequences then being amplified and cloned by the PCR procedure to yield clones encoding proteins or peptides having at least partial homology to said sequences of the invention.

The present invention also provides a pharmaceutical composition for the modulation of the expression, proteolytic processing, bioactivity or intracellular signaling of the 26 kDa TNF comprising, as active ingredient, a modulator of the invention, and a pharmaceutically acceptable excipient, carrier or diluent.

Moreover, the present invention also provides the following pharmaceutical compositions:
(i) a pharmaceutical composition for the modulation of the expression, proteolytic processing, bioactivity or intracellular signaling of the 26 kDa TNF comprising, as active ingredient, a recombinant animal virus vector encoding a protein capable of binding a cell surface receptor and encoding a protein or peptide or analogs thereof of the invention;
(ii) a pharmaceutical composition for the modulation of the expression, proteolytic processing, bioactivity or intracellular signaling of the 26 kDa TNF comprising, as active ingredient, an oligonucleotide sequence encoding an anti-sense sequence of the DNA sequence of the invention.

In addition, the present invention also provides a method for designing drugs that are capable of modulating the expression, proteolytic processing, bioactivity or intracellular signaling of the 26 kDa TNF comprising the procedures described herein in Examples 6 and 7.

Other aspects and embodiments of the invention are also provided as arising from the following detailed description of the invention.

It should be noted that, where used throughout, the term “modulation of the expression, proteolytic processing, bioactivity or intracellular signaling of the 26 kDa TNF” is understood to encompass in vitro as well as in vivo treatment.

Moreover, where used throughout, the antibodies of the invention and methods using these antibodies, include so-called “humanized” antibodies or the use thereof.

### BRIEF DESCRIPTION OF THE FIGURES

FIGS. 1A and B show the cell-surface TNF in HeLa-M9 cells and in LPS-treated MM6 cells, wherein FIG. 1A shows a graphic representation of the flow cytometric analysis data of cell-surface TNF expression in HeLa, HeLa-M9 and LPS-treated MM6 cells; and in FIG. 1B there is shown the FACS profiles of HeLa and HeLa-M9 cells stained with anti-TNF antibodies, all as described in Example 1.

FIGS. 2A-D show the SDS-PAGE and Western blotting analysis of TNF expressed in HeLa-M9 and LPS-treated MM6 cells, wherein FIG. 2A shows a reproduction of an autoradiogram of an SDS-PAGE gel on which were separated, as test samples, proteins immunoprecipitated with anti-TNF antibody from [³⁵S]-Met metabolically labeled HeLa-M9 cells either before or after lysis of the cells; FIG. 2B shows a reproduction of an autoradiogram of a Western blot of proteins in the lysate of HeLa-M9 cells that react with anti-TNF antibody; FIG. 2C shows an autoradiogram of an SDS-PAGE gel on which were separated, as test samples, proteins immunoprecipitated with anti-TNF antibody from [³⁵S]-Met metabolically-labeled MM6 cells that were treated with LPS or were untreated: and FIG. 2D shows a reproduction of a Western blot of proteins in the lysates of MM6 cells (LPS-treated or untreated) that react with anti-TNF antibodies all as described in Example 2.

FIGS. 3A and B show the phosphorvlation of the 26 kDa TNF molecules in HeLa-M9 and LPS-treated MM6 cells, wherein FIG. 3A shows the reproduction of an autoradiogram of an SDS-PAGE gel on which were separated [³²P]-labeled proteins from HeLa-M9 cells which were immunoprecipitated before or after cell lysis with anti-TNF antibodies; and FIG. 3B shows a reproduction of an autoradiogram of an SDS-PAGE gel on which were separated [³²P]-labeled proteins from MM6 cells (LPS-treated or untreated) that were immunoprecipitated with anti-TNF antibodies, all as described in Example 3.

FIG. 4 shows the phosphoamino acid analysis of the 26 kDa TNF by way of a reproduction of an autoradiogram of a two-dimensional thin layer electrophoretic separation of phosphoamino acids obtained by immunoprecipitation of TNF by anti-TNF antibodies from lysates of [³²P]-labeled HeLa-M9 cells followed by hydrolysis of the immunoprecipitated proteins and their subjection to the two-dimensional thin layer electrophoresis, as described in Example 4.

### DETAILED DESCRIPTION OF THE INVENTION

In accordance with the invention there were employed cellular systems that provide effective expression of the membrane bound form of TNF, to allow study of the molecular properties of this protein which is normally present in very low amounts (see Examples 14). The MM6 monocytic leukemia cells were chosen since, in contrast to some other cultured cells of monocytic origin, LPS-stimulated TNF production in them is not accompanied by induced TNF shedding. Thus, the transmembrane form of TNF is effectively accumulated in these cells (Pardines-Figueres et al., 1992). Indeed, 26 kDa TNF molecules were easily detected in lysates of LPS-treated MM6 cells. However, the amounts of cell-surface TNF molecules in these cells were too low to allow their detection by metabolic labeling (although they could be detected by FACS analysis). We therefore decided to use an artificial experimental system where TNF was expressed in HeLa cells under the control of a strong promoter. To further enhance the expression of the precursor TNF molecules, we used a mutant TNF molecule that cannot be processed effectively. The change introduced by the mutation (substitution of the arginine and serine at positions +2 and +3 with threonines) was milder than applied in a previous study (deletion of amino acids 1-12 in TNF [Perez et al., 1990]), to minimize distortion of normal TNF function. This change does not fully prevent the proteolytic cleavage of TNF, but it does result in the accumulation of 26 kDa TNF molecules, both intracellularly and on the cell surface.

We found that the 26 kDa TNF molecules are phosphorylated. The high amounts of TNF in the transfected HeLa cells permitted further studies in which we found that (i) both the cell surface and intracellular 26 kDa TNF molecules are phosphorylated, (ii) the phosphorylated residues in TNF are serines, and (iii) the soluble 17 kDa TNF molecules are not phosphorylated. Such analysis could not be performed with 26 kDa TNF molecules from MM6 cells, due to the low amounts of TNF present. However, the mere finding that the 26 kDa molecules are also phosphorylated in these cells is significant; it shows that phosphorylation is not an artifact of the expression of TNF in the HeLa cells, that normally produce little TNF, but rather constitutes part of the normal way of TNF modulation.

The lack of [³²p] incorporation in the 17 kDa TNF molecules isolated from the lysate of the HeLa-M9 cells, indicates that the label in the 26 kDa molecules occurs within their intracellular region. The intracellular region is the only part of the TNF molecule accessible for phosphorylation by cytoplasmic protein kinases. The specific kinases involved in TNF phosphorylation are not known, nor is it known if, and in what way, the activity of these kinases is subject to modulation by agents that affect TNF activity. Evidently, the phosphorylation observed in the HeLa-M9 cells, in which TNF was synthesized without stimulation, reflects the function of kinase(s) that constitutively act in these cells. On the other hand, the phosphorylation observed in the LPS-stimulated MM6 cells could involve effects of LPS activated protein kinases (Liu et al., 1994; Han et al., 1995). The serine at position −50 seems to be a suitable substrate for phosphorylation by protein kinase C (Kennelly and Krebs, 1991). However, in preliminary experiments, we did not observe any increase of phosphorylation of the 26 kDa TNF molecules in HeLa-M9 cells following treatment with 4β-phorbol-12-myristate-13-acetate (data not shown), suggesting that protein kinase C is either not involved in this phosphorylation or is activated constitutively in these cells, due to their continuous exposure to TNF.

The sequence conservation of the cytoplasmic region of the TNF molecule in different species indicates that this region and its phosphorylation play important roles in TNF function. Several possible kinds of roles can be considered. One possibility is that this region takes part in the regulation of the proteolytic process by which the soluble 17 kDa form of TNF is derived from the 26 kDa molecule. Involvement of the intracellular region of transmembrane proteins in the regulation of their shedding has been observed for certain proteins. This seems to be the case for the processing of TGF-α, which, like TNF, is expressed initially as a transmembrane protein (Bosenberg et al., 1992), as well as for the induced shedding of the p75 TNF receptor (Crowe et al., 1993). In contrast thereto, shedding of the p55 TNF receptor appears to be independent of the intracellular domain of this receptor (Brakebusch et al., 1992, Brakebusch et al., 1994). The intracellular domain of cell surface TNF may also affect TNF function as a ligand. This region in the molecule may impose conformational changes in the ligand binding of the extracellular TNF domain, or could dictate association with cytoskeletal elements, and thus direct translocation of the TNF molecules within the membrane towards the area of the cell surface adjacent to the target cell. The intracellular region of the Fas ligand, whose structure and activity closely resemble those of TNF, is indeed known to contain a sequence motif, the SH3 binding site, that may allow it to bind to cytoskeletal components (Takahashi et al., 1994). Another possible function of the cytoplasmic region of TNF is interaction with intracellular molecules possessing signaling activities. Activation of signaling activities within the TNF producing cell following the interaction of the cell surface TNF with its target cell may allow fine adjustment of the function or formation of the cell surface TNF molecules, depending on the situation.

Further studies of the phosphorylation of the cytoplasmic TNF domain may contribute not only to our knowledge of the cell surface form of this particular cytokine, but also to our understanding of the mode of action of some other cell surface ligands that are evolutionarily related to TNF, including the CD40 ligand (gp39), the OX-40 ligand (the human activation antigen 106, gp34), 4-IBB and the ligands for CD27, CD30 and for Fas/APO1 (reviewed in [Bazan, 1993]).

Thus, in accordance with the present invention, the finding of phosphorylation of the intracellular domain of the cell-surface-bound form of TNF provides a basis for isolating agents on the one hand, and for pinpointing agents on the other, that can: (i) modulate the shedding (or proteolytic processing) of TNF, i.e. the release of the 17 kDa soluble form of TNF from the membrane-bound 26 kDa form; (ii) modulate the activity of TNF via intracellular signaling induced by the intracellular domain of TNF; or (iii) modulate the bioactivity of TNF via conformational interactions mediated by the intracellular domain.

Further, the location of the site of phosphorylation in the intracellular domain of TNF provides a “handle” on the way to approach the modulation of the activity of the membrane-bound form of TNF. In this respect, recent data indicates that the nature of the function of cell-surface associated TNF is qualitatively different to the soluble TNF, for example, cell-surface (26 kDa) TNF stimulates p75 TNF-R at a much higher level than that observed for soluble TNF. Moreover, TNF is one of the only members of the TNF/NGF family that occurs in a soluble form, most, if not all, of the others are in cell-surface associated form and are biologically active in this way, i.e. activity is between the cell carrying the ligand (e.g. TNF or FAS/APO1 ligand) and the cell carrying the receptor (e.g. TNF-R or FAS/APOI), or in some cases autoregulation of the same cell occurs, i.e. cells which express both the ligand and the receptor can be induced to self-destruct by binding of the ligand to the receptor at the cell-surface, for example, in the case of autoregulation of cells carrying Fas/APO1 ligand and receptor. In other cases, for example, macrophages, there is possibly an autoregulatory process mediated by TNF in which these cells have both a cell-surface form of TNF and TNF-Rs with the result that there can occur binding between the TNF and TNF-Rs at the cell surface, which may not necessarily kill the cells but which can influence the amount of TNF production in these cells possibly via signals mediated by the intracellular domain of TNF. This form of autoregulation is considered as playing an important role in both the level of TNF production by the macrophages and in the differentiation of the macrophages.

The present invention therefore concerns, in one aspect, modulators of the expression, proteolytic processing, bioactivity, or intracellular signaling of the 26 kDa TNF, which modulators are capable of interacting with the intracellular domain of the 26 kDa TNF or with one or more other effector proteins which interact with the intracellular domain of the 26 kDa TNF. These modulators can be any of the following group: (i) naturally-derived proteins, peptides, analogs and derivatives thereof capable of interacting with the intracellular domain of 26 kDa TNF or with the other intracellular effect proteins; (ii) synthetically produced complementary peptides synthesized by using as substrate the intracellular domain or portions thereof of the 26 kDa TNF, the complementary peptides being capable of interacting with the intracellular domain of the 26 kDa TNF or with the other intracellular effector proteins; (iii) antibodies or active fragments thereof capable of interacting with the intracellular domain of the 26 kDa TNF or with the other intracellular effector proteins; and (iv) organic compounds capable of interacting with the intracellular domain of the 26 kDa TNF or with the other intracellular effector proteins, the organic compounds being derived from known compounds and selected using the intracellular domain or portions thereof of 26 kDa TNF as a substrate in a binding assay, or being synthesized using the intracellular domain or portions thereof of 26 kDa TNF as a substrate for designing and synthesizing the organic compounds.

Moreover, the modulators of the invention include those which are capable of interacting with one or more of the serine residues present in the intracellular domain of the 26 kDa TNF, which serines are the substrate for the observed phosphorylation of the 26 kDa TNF, and in this way represent a group of modulators which specifically modulate the phosphorylation of the 26 kDa TNF and hence its bioactivity, proteolytic processing, level of expression, or intracellular signaling activity.

When the modulators of the invention are proteins or peptides, they may be obtained as described in the above noted co-pending application nos. IL 109632, 111125, 112002 and 112742 (see also Example 5), by use of the yeast two-hybrid procedure in which the intracellular domain or portions thereof of the 26 kDa TNF will be used as probes or “baits” to isolate from genomic or cDNA libraries, clones expressing proteins or peptides capable of binding to the intracellular domain of the 26 kDa TNF.

Other approaches for obtaining the above proteins and peptides of the invention include the well known standard procedures such as, for example, affinity chromatography in which, for example, the intracellular domain of the 26 kDa TNF or portions thereof including the portion with the one or more serines which undergo phosphorylation, are attached to the chromatography substrate or matrix and are brought into contact with cell extracts or lysates (of human/mammalian origin) and thereby proteins or peptides are isolated which are capable of binding to the intracellular domain or portions thereof of the 26 kDa TNF. Likewise, other standard chemical and recombinant DNA procedures usually employed for isolating proteins or peptides capable of binding to a specific amino acid sequence (26 kDa TNF intracellular domain sequence) can be employed to obtain these proteins and peptides of the invention.

Thus, the present invention also concerns the DNA sequences encoding the proteins and peptides of the invention and the proteins and peptides encoded by these sequences.

Moreover, the present invention also concerns the DNA sequences encoding biologically active analogs and derivatives of these proteins and peptides of the invention, and the analogs and derivatives encoded thereby. The preparation of such analogs and derivatives is by standard procedure (see for example, Sambrook et al., 1989) in which in the DNA sequences encoding these proteins, one or more codons may be deleted, added or substituted by another, to yield analogs having at least a one amino acid residue change with respect to the native protein. Acceptable analogs are those which retain at least the capability of binding to the intracellular domain or portions thereof of the 26 kDa TNF, or which can mediate any other binding or enzymatic activity, e.g. analogs which bind the intracellular domain of the 26 kDa TNF but which do not signal, i.e. do not bind to a further downstream receptor, protein or other factor, or do not catalyze a signal-dependent reaction. In such a way analogs can be produced which have a so-called dominant-negative effect, namely, an analog which is defective either in binding to the 26 kDa intracellular domain or in subsequent signaling following such binding. Such analogs can be used, for example, to inhibit the TNF effect on cells by competing with the natural 26 kDa TNF intracellular domain-binding proteins (e.g. kinases) which are necessary for normal 26 kDa TNF activity.

Likewise, so-called dominant-positive analogs may be produced which would serve to enhance, for example, the TNF effect on cells. These would have the same or better 26 kDa TNF intracellular domain-binding properties and the same or better signaling properties of the natural 26 kDa TNF intracellular domain-binding proteins. Similarly, derivatives may be prepared by standard modifications of the side groups of one or more amino acid residues of the proteins or peptides, or by conjugation of the proteins or peptides to another molecule e.g. an antibody, enzyme, receptor, etc., as are well known in the art.

The new 26 kDa TNF intracellular domain-binding proteins and peptides of the invention have a number of possible uses, for example:
(i) They may be used to enhance the function of TNF in situations where such an enhanced effect is desired such as in anti-tumor, anti-inflammatory, anti-septic shock or other disease/disorder applications where the enhanced activity is desired. In this case the proteins or peptides may be introduced into the cells by standard procedures known per se. For example, as the proteins or peptides are required to act intracellularly, i.e. bind/interact with intracellularly located 26 kDa TNF intracellular domain and it is desired that they be introduced only into the cells where their effect is wanted, a system for specific introduction of these proteins or peptides into the cells is necessary. One way of doing this is by creating a recombinant animal virus e.g. one derived from Vaccinia, to the DNA of which the following two genes will be introduced: the gene encoding a ligand that binds to cell surface proteins specifically expressed by the cells e.g. ligands specific to receptors carried by TNF-producing cells such as macrophages, such that the recombinant virus vector will be capable of binding such cells, and the gene encoding the new 26 kDa TNF intracellular domain-binding protein or peptide. Thus, expression of the cell-surface-binding protein on the surface of the virus will target the virus specifically to the TNF-producing cells, following which the 26 kDa intracellular domain-binding protein or peptide encoding sequence will be introduced into the cells via the virus, and once expressed in the cells will result in enhancement of, for example, the proteolytic processing of TNF to yield more soluble TNF, the bioactivity of TNF, or the expression of TNF leading to, for example, enhanced TNF-mediated death of the tumor cells or other cells it is desired to kill. Construction of such recombinant animal virus is by standard procedures (see for example, Sambrook et al., 1989). Another possibility is to introduce the sequences of the new proteins or peptides in the form of oligonucleotides which can be absorbed by the cells and expressed therein.
(ii) They may be used to inhibit the function of TNF, e.g. in cases such as tissue damage in septic shock, graft-vs.-host rejection, or other diseases/disorders in which case it is desired to block the TNF-induced cellular effects. In this situation it is possible, for example, to introduce into the cells, by standard procedures, oligonucleotides having the anti-sense coding sequence for these new proteins or peptides which would effectively block the translation of mRNAs encoding these proteins and thereby block their expression and lead to the desired inhibition in the proteolytic processing, expression, bioactivity or intracellular signaling mediated by the intracellular domain of the 26 kDa TNF and hence reduction in the overall activity of TNF.
Such oligonucleotides may be introduced into the cells using the above recombinant virus approach, the second sequence carried by the virus being the oligonucleotide sequence. Another possibility is to use antibodies specific for these proteins or peptides to inhibit their intracellular activity (via their binding to the intracellular domain of the 26 kDa TNF).
Yet another way of inhibiting the TNF effect on cells is by the recently developed ribozyme approach. Ribozymes are catalytic RNA molecules that specifically cleave RNAs. Ribozymes may be engineered to cleave target RNAs of choice, e.g. the mRNAs encoding the new proteins or peptides of the invention. Such ribozymes would have a sequence specific for the mRNA of choice and would be capable of interacting therewith (complementary binding) followed by cleavage of the mRNA, resulting in a decrease (or complete loss) in the expression of the protein or peptide it is desired to inhibit, the level of decreased expression being dependent upon the level of ribozyme expression in the target cell. In this way, when such proteins or peptides are essential for mediating the normal proteolytic processing, bioactivity, expression or intracellular signaling by binding to the intracellular domain of the 26 kDa TNF, the ribozyme-mediated inhibition of these proteins or peptides will thus result in reduced TNF biological activity. To introduce ribozymes into the cells of choice any suitable vector may be used, e.g. plasmid, animal virus (retrovirus) vectors, that are usually used for this purpose (see also (i) above, where the virus has, as second sequence, a cDNA encoding the ribozyme sequence of choice). Moreover, ribozymes can be constructed which have multiple targets (multi-target ribozymes) that can be used, for example, to inhibit the expression of one or more of the proteins or peptides of the invention (For reviews, methods etc. concerning ribozymes see Chen et al., 1992; Zhao and Pick, 1993; Shore et al., 1993; Joseph and Burke, 1993; Shimayama et al., 1993; Cantor et al., 1993; Barinaga, 1993; Crisell et al., 1993 and Koizumi et al., 1993).
(iii) They may be used to isolate, identify and clone yet other proteins or peptides which are capable of binding to them, e.g. other proteins or peptides involved in the intracellular signaling process, proteolytic processing, expression or bioactivity of TNF that are downstream of the 26 kDa TNF intracellular domain-binding proteins or peptides. In this situation, these options, namely, the DNA sequences encoding them may be used in the yeast two-hybrid system (see Example 5 below) in which the sequence of these proteins or peptides will be used as “baits” to isolate, clone and identify from cDNA or genomic DNA libraries other sequences (“preys”) encoding proteins which can bind to these new 26 kDa TNF intracellular domain-binding proteins. In the same way, it may also be determined whether the specific proteins or peptides of the present invention, namely, those which bind to the intracellular domain of the 26 kDa TNF, can bind to yet other receptors or proteins. Moreover, this approach may also be taken to determine whether the proteins or peptides of the present invention are capable of binding to other known receptors or proteins in whose activity they may have a functional role, i.e. other as yet unidentified receptors or proteins, sharing homology with the intracellular domain of the 26 kDa TNF, e.g. other members of the TNF/NGF family.
(iv) The new proteins may also be used to isolate, identify and clone other proteins of the same class i.e. those binding to the 26 kDa TNF intracellular domain or to functionally related receptors or proteins, and involved in their modulation/mediation. In this application, the above noted yeast two-hybrid system may be used, or there may be used a recently developed (Wilks et al., 1989) system employing non-stringent southern hybridization followed by PCR cloning. In the Wilks et al. publication, there is described the identification and cloning of two putative protein-tyrosine kinases by application of non-stringent southern hybridization followed by cloning by PCR based on the known sequence of the kinase motif, a conserved kinase sequence. This approach may be used, in accordance with the present invention using the sequences of the new proteins or peptides to identify and clone those of related 26 kDa TNF intracellular domain-binding proteins or peptides also capable of binding to the 26 kDa TNF intracellular domain.
(v) Yet another approach to utilizing the new proteins of the invention is to use them in methods of affinity chromatography to isolate and identify yet other proteins or factors to which they are capable of binding as noted above. In this application, the proteins of the present invention, may be individually attached to affinity chromatography matrices and then brought into contact with cell extracts or isolated proteins or factors suspected of being involved in the modulation of the 26 kDa TNF. Following the affinity chromatography procedure, the other proteins or factors which bind to the new proteins of the invention, can be eluted, isolated and characterized.
(vi) As noted above, the new proteins or peptides of the invention may also be used as immunogens (antigens) to produce specific antibodies thereto. These antibodies may also be used for the purposes of purification of the new proteins or peptides either from cell extracts or from transformed cell lines producing them. Further, these antibodies may be used for diagnostic purposes for identifying disorders related to abnormal functioning of, for example, the TNF system, e.g. overactive or underactive TNF. Thus, should such disorders be related to a malfunctioning intracellular signaling or other regulation system controlling TNF expression and activity that is mediated by the new proteins or peptides, such antibodies would serve as an important diagnostic tool.

In another aspect, the present invention relates to the above mentioned modulators of the invention, when these are complementary peptides. These complementary peptides of the invention may be synthesized by well known standard procedures of the art, that are capable of binding or interacting specifically with the intracellular domain or portions thereof of the 26 kDa TNF. These complementary peptides will be synthesized using, for example, the 26 kDa intracellular domain of portions thereof, as substrates and synthesizing by standard chemical means peptides of sequence that are complementary to these 26 kDa TNF intracellular domain sequences. A suitable complementary peptide is one that will be capable of binding to one or more of these 26 kDa TNF intracellular domain or portions thereof and thereby being capable of modulating or mediating the activity of the 26 kDa TNF, via modulation of the expression, proteolytic processing, intracellular signaling or bioactivity of the 26 kDa TNF.

The so-generated complementary peptides, and likewise, DNA sequences encoding them, which may be readily produced by standard procedures, may be employed, as noted above in any one of uses (i)-(vi), i.e. to enhance (gain-of-function) or inhibit the activity of the 26 kDa TNF, or may be used to generate specific antibodies thereto for modulation/mediation, isolation or diagnostic purposes.

It should also be noted that included in the present invention are the antibodies (and their uses) specific to the proteins and peptides of the invention including the complementary peptides, as well as antibodies specific to the intracellular domain of the 26 kDa TNF or portions thereof These antibodies may be used for directly modulating/mediating the activity of the 26 kDa TNF in the ways noted above or for isolation, identification and characterization (including diagnostic applications, as noted above) of other proteins and receptors as also noted above.

As regards the antibodies mentioned herein throughout, the term “antibody” is meant to include polyclonal antibodies, monoclonal antibodies (mAbs), chimeric antibodies, anti-idiotypic (anti-Id) antibodies to antibodies that can be labeled in soluble or bound form, as well as fragments thereof provided by any known technique, such as, but not limited to enzymatic cleavage, peptide synthesis or recombinant techniques.

Polyclonal antibodies are heterogeneous populations of antibody molecules derived from the sera of animals immunized with an antigen. A monoclonal antibody contains a substantially homogeneous population of antibodies specific to antigens, which populations contains substantially similar epitope binding sites. MAbs may be obtained by methods known to those skilled in the art. See, for example Kohler and Milstein, Nature, 256:495-497 (1975); U.S. Pat. No. 4,376,110; Ausubel et al., eds., Harlow and Lane ANTIBODIES: A LABORATORY MANUAL, Cold Spring Harbor Laboratory (1988); and Colligan et al., eds., Current Protocols in Immunology, Greene publishing Assoc. and Wiley Interscience N.Y., (1992, 1993), the contents of which references are incorporated entirely herein by reference. Such antibodies may be of any immunoglobulin class including IgG, IgM, IgE, IgA, GILD and any subclass thereof. A hybridoma producing a mAb of the present invention may be cultivated in vitro, in situ or in vivo. Production of high titers of mAbs in vivo or in situ makes this the presently preferred method of production.

Chimeric antibodies are molecules different portions of which are derived from different animal species, such as those having the variable region derived from a murine mAb and a human immunoglobulin constant region. Chimeric antibodies are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art (Cabilly et al., Proc. Natl. Acad Sci. USA 81:3273-3277 (1984); Morrison et al., Proc. Natl. Acad Sci. USA 81:6851-6855 (1984); Boulianne et al., Nature 312:643-646 (1984); Cabilly et al., European Patent Application 125023 (published Nov. 14, 1984); Neuberger et al., Nature 314:268-270 (1985); Taniguchi et al., European Patent Application 171496 (published Feb. 19, 1985); Morrison et al., European Patent Application 173494 (published Mar. 5, 1986); Neuberger et al., PCT Application WO 8601533, (published Mar. 13, 1986); Kudo et al., European Patent Application 184187 (published Jun. 11, 1986); Sahagan et al., J. Immunol. 137:1066-1074 (1986); Robinson et al., International Patent Application No. WO8702671 (published May 7, 1987); Liu et al., Proc. Natl. Acad. Sci USA 84:3439-3443 (1987); Sun et al., Proc. Natl. Acad Sci USA 84:214-218 (1987); Better et al., Science 240:1041-1043 (1988); and Harlow and Lane, ANTIBODIES: A LABORATORY MANUAL, supra. These references are entirely incorporated herein by reference.

An anti-idiotypic (anti-Id) antibody is an antibody which recognizes unique determinants generally associated with the antigen-binding site of an antibody. An Id antibody can be prepared by immunizing an animal of the same species and genetic type (e.g. mouse strain) as the source of the mAb with the mAb to which an anti-Id is being prepared. The immunized animal will recognize and respond to the idiotypic determinants of the immunizing antibody by producing an antibody to these idiotypic determinants (the anti-Id antibody). See, for example, U.S. Pat. No. 4,699,880, which is herein entirely incorporated by reference.

The anti-Id antibody may also be used as an “immunogen” to induce an immune response in yet another animal, producing a so-called anti-anti-Id antibody. The anti-anti-Id may be epitopically identical to the original mAb which induced the anti-Id. Thus, by using antibodies to the idiotypic determinants of a mAb, it is possible to identify other clones expressing antibodies of identical specificity.

Accordingly, mabs generated against the 26 kDa TNF intracellular domain or portions thereof, 26 kDa TNF intracellular domain-binding proteins or peptides, or 26 kDa TNF intracellular domain-binding complementary peptides, analogs or derivatives thereof of the invention may be used to induce anti-Id antibodies in suitable animals, such as BALB/c mice. Spleen cells from such immunized mice are used to produce anti-Id hybridomas secreting anti-Id mAbs. Further, the anti-Id mAbs can be coupled to a carrier such as keyhole limpet hemocyanin (KLH) and used to immunize additional BALB/c mice. Sera from these mice will contain anti-anti-Id antibodies that have the binding properties of the original mAb specific for an epitope of the above proteins, peptides, analogs or derivatives.

The anti-Id mAbs thus have their own idiotypic epitopes, or “idiotopes” structurally similar to the epitope being evaluated, such as GRB protein-α.

The term “antibody” is also meant to include both intact molecules as well as fragments thereof, such as, for example, Fab and F(ab′)₂, which are capable of binding antigen. Fab and F(ab′)₂ fragments lack the Fc fragment of intact antibody, clear more rapidly from the circulation, and may have less non-specific tissue binding than an intact antibody (Wahl et al., J. Nucl. Med 24:316-325 (1983)).

It will be appreciated that Fab and F(ab′)₂ and other fragments of the antibodies useful in the present invention may be used for the detection and quantitation of the 26 kDa TNF intracellular domain-binding proteins or peptides according to the methods disclosed herein for intact antibody molecules. Such fragments are typically produced by proteolytic cleavage, using enzymes such as papain (to produce Fab fragments) or pepsin (to produce F(ab′)₂ fragments).

An antibody is said to be “capable of binding” a molecule if it is capable of specifically reacting with the molecule to thereby bind the molecule to the antibody. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

An “antigen” is a molecule or a portion of a molecule capable of being bound by an antibody which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens.

The antibodies, including fragments of antibodies, useful in the present invention may be used to quantitatively or qualitatively detect the 26 kDa TNF intracellular domain-binding proteins or peptides (including complementary peptides) in a sample or to detect presence of cells which express the 26 kDa TNF intracellular domain-binding proteins or peptides of the present invention. This can be accomplished by immunofluorescence techniques employing a fluorescently labeled antibody (see below) coupled with light microscopic, flow cytometric, or fluorometric detection.

The antibodies (or fragments thereof) useful in the present invention may be employed histologically, as in immunofluorescence or immunoelectron microscopy, for in situ detection of 26 kDa TNF intracellular domain-binding proteins or peptides of the present invention. In situ detection may be accomplished by removing a histological specimen from a patient, and providing the labeled antibody of the present invention to such a specimen. The antibody (or fragment) is preferably provided by applying or by overlaying the labeled antibody (or fragment) to a biological sample. Through the use of such a procedure, it is possible to determine not only the presence of the 26 kDa TNF intracellular domain-binding proteins or peptides, but also its distribution on the examined tissue. Using the present invention, those of ordinary skill will readily perceive that any of wide variety of histological methods (such as staining procedures) can be modified in order to achieve such in situ detection.

Such assays for 26 kDa TNF intracellular domain-binding proteins of the present invention typically comprise incubating a biological sample, such as a biological fluid, a tissue extract, freshly harvested cells such as lymphocytes or leukocytes, or cells which have been incubated in tissue culture, in the presence of a detectably labeled antibody capable of identifying the 26 kDa TNF intracellular domain-binding proteins or peptides, and detecting the antibody by any of a number of techniques well known in the art.

The biological sample may be treated with a solid phase support or carrier such as nitrocellulose, or other solid support or carrier which is capable of immobilizing cells, cell particles or soluble proteins. The support or carrier may then be washed with suitable buffers followed by treatment with a detectably labeled antibody in accordance with the present invention, as noted above. The solid phase support or carrier may then be washed with the buffer a second time to remove unbound antibody. The amount of bound label on said solid support or carrier may then be detected by conventional means.

By “solid phase support”, “solid phase carrier”, ”solid support”, “solid carrier”, “support” or “carrier” is intended any support or carrier capable of binding antigen or antibodies. Well-known supports or carriers, include glass, polystyrene, polypropylene, polyethylene, dextran, nylon amylases, natural and modified celluloses, polyacrylamides, gabbros and magnetite. The nature of the carrier can be either soluble to some extent or insoluble for the purposes of the present invention. The support material may have virtually any possible structural configuration so long as the coupled molecule is capable of binding to an antigen or antibody. Thus, the support or carrier configuration may be spherical, as in a bead, cylindrical, as in the inside surface of a test tube, or the external surface of a rod. Alternatively, the surface may be flat such as a sheet, test strip, etc. Preferred supports or carriers include polystyrene beads. Those skilled in the art will know may other suitable carriers for binding antibody or antigen, or will be able to ascertain the same by use of routine experimentation.

The binding activity of a given lot of antibody, of the invention as noted above, may be determined according to well known methods. Those skilled in the art will be able to determine operative and optimal assay conditions for each determination by employing routine experimentation.

Other such steps as washing, stirring, shaking, filtering and the like may be added to the assays as is customary or necessary for the particular situation.

One of the ways in which an antibody in accordance with the present invention can be detectably labeled is by linking the same to an enzyme and use in an enzyme immunoassay (EIA). This enzyme, in turn, when later exposed to an appropriate substrate, will react with the substrate in such a manner as to produce a chemical moiety which can be detected, for example, by spectrophotometric, fluorometric or by visual means. Enzymes which can be used detectably label the antibody include, but are not limited to, malate dehydrogenase, staphylococcal nuclease, delta-5-steroid isomeras, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetyl-cholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may be accomplished using any of a variety of other immunoassays. For example, by radioactivity labeling the antibodies or antibody fragments, it is possible to detect R-PTPase through the use of a radioimmunoassay (RIA). A good description of RIA may be found in Laboratory Techniques and Biochemistry in Molecular Biology, by Work, T. S. et al., North Holland Publishing Company, NY (1979) with particular reference to the chapter entitled “An Introduction to Radioimmune Assay and Related Techniques” by Chard, T., incorporated by reference herein. The radioactive isotope can be detected by such means as the use of a y counter or a scintillation counter or by autoradiography.

It is also possible to label an antibody in accordance with the present invention with a fluorescent compound. When the fluorescently labeled antibody is exposed to light of the proper wavelength, its presence can be then detected due to fluorescence. Among the most commonly used fluorescent labeling compounds are fluorescein isothiocyanate, rhodamine, phycoerythrine, pycocyanin, allophycocyanin, o-phthaldehyde and fluorescarnine.

The antibody can also be detectably labeled using fluorescence emitting metals such as ¹⁵²E, or others of the lanthanide series. These metals can be attached to the antibody using such metal chelating groups as diethylenetriamine pentaacetic acid (ETPA).

The antibody can also be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

An antibody molecule of the present invention may be adapted for utilization in an immunometric assay, also known as a “two-site” or “sandwich” assay. In a typical immunometric assay, a quantity of unlabeled antibody (or fragment of antibody) is bound to a solid support or carrier and a quantity of detectably labeled soluble antibody is added to permit detection and/or quantitation of the ternary complex formed between solid-phase antibody, antigen, and labeled antibody.

Typical, and preferred, immunometric assays include “forward” assays in which the antibody bound to the solid phase is first contacted with the sample being tested to extract the antigen from the sample by formation of a binary solid phase antibody-antigen complex. After a suitable incubation period, the solid support or carrier is washed to remove the residue of the fluid sample, including unreacted antigen, if any, and the contacted with the solution containing an unknown quantity of labeled antibody (which functions as a “reporter molecule”). After a second incubation period to permit the labeled antibody to complex with the antigen bound to the solid support or carrier through the unlabeled antibody, the solid support or carrier is washed a second time to remove the unreacted labeled antibody.

In another type of “sandwich” assay, which may also be useful with the antigens of the present invention, the so-called “simultaneous” and “reverse” assays are used. A simultaneous assay involves a single incubation step as the antibody bound to the solid support or carrier and labeled antibody are both added to the sample being tested at the same time. After the incubation is completed, the solid support or carrier is washed to remove the residue of fluid sample and uncomplexed labeled antibody. The presence of labeled antibody associated with the solid support or carrier is then determined as it would be in a conventional “forward” sandwich assay.

In the “reverse” assay, stepwise addition first of a solution of labeled antibody to the fluid sample followed by the addition of unlabeled antibody bound to a solid support or carrier after a suitable incubation period is utilized. After a second incubation, the solid phase is washed in conventional fashion to free it of the residue of the sample being tested and the solution of unreacted labeled antibody. The determination of labeled antibody associated with a solid support or carrier is then determined as in the “simultaneous” and “forward” assays.

The new proteins and peptides of the invention once isolated, identified and characterized by any of the standard screening procedures for example, the yeast two-hybrid method, affinity chromatography, and any other well known method known in the art, may then be produced by any standard recombinant DNA procedure (see for example, Sambrook, et al., 1989) in which suitable eukaryotic or prokaryotic host cells are transformed by appropriate eukaryotic or prokaryotic vectors containing the sequences encoding for the proteins.

Accordingly, the present invention also concerns such expression vectors and transformed hosts for the production of the proteins of the invention. As mentioned above, these proteins also include their biologically active analogs and derivatives, and thus the vectors encoding them also include vectors encoding analogs of these proteins, and the transformed hosts include those producing such analogs. The derivatives of these proteins are the derivatives produced by standard modification of the proteins or their analogs, produced by the transformed hosts.

In yet another aspect of the invention there is provided the modulators of the invention when these are organic compounds, e.g. heterocyclic compounds, which are capable of specifically binding to the intracellular domain of the 26 kDa TNF. These organic compounds are well known in the field of pharmaceuticals and are widely used as therapeutic agents which are capable of entering cells (hydrophobic/lipophilic compounds) and binding various intracellular proteins or intracellular portions of transmembrane proteins and thereby exerting their effect. These organic compounds may be readily screened and identified by using the intracellular domain or portions thereof of the 26 kDa TNF, in standard affinity chromatography procedures or other methods well known in the art.

The present invention also relates to pharmaceutical compositions containing as active ingredient, one or more of the modulators of the invention. For example, these compositions include those comprising one or more of the 26 kDa TNF intracellular domain-binding proteins, peptides, analogs or derivatives thereof, or antibodies specific to the 26 kDa TNF intracellular domain or the above proteins, peptides, or analogs; or recombinant animal virus vectors encoding the 26 kDa TNF intracellular domain-binding proteins or peptides, which vector also encodes a virus surface protein capable of binding specific target cell (e.g. TNF-producing cell) surface proteins to direct the insertion of the 26 kDa TNF intracellular domain-binding protein or peptide sequences into the cells. Likewise, the present invention also relates to pharmaceutical compositions comprising organic compounds capable of binding to the 26 kDa TNF intracellular domain.

The way of administration can be via any of the accepted modes of administration for similar agents and will depend on the condition to be treated, e.g. administration may be intravenously, or continuously by infusion, etc.

The pharmaceutical compositions of the invention are prepared for administration by mixing the active ingredient or its derivatives with physiologically acceptable carriers, stabilizers and excipients, and prepared in dosage form, e.g. by lyophilization in dosage vials. The amount of active compound to be administered will depend on the route of administration, the disease to be treated and the condition of the patient.

The present invention will now be described in more detail in the following non-limiting Examples and the accompanying figures

General Procedures and Materials:

(a) Reagents:

Cell culture media and supplements were purchased from GIBCO, Grand Island, N.Y.; bovine insulin, lipopolysaccharide (LPS, obtained from the bacterial strain Salmonella Minnesota, phenylmethylsulfonyl fluoride (PMSF), leupeptin, and diaminobenzidine-tetrahydrochloride were purchased from Sigma. Chemical Co., St. Louis, Mo., U.S.A.; Protein G-Sepharose (fast flow) were purchased from Pharmacia Fine Chemicals, Piscataway, N.J., U.S.A; and the radiolabeled reagents [³⁵S]methionine ([³⁵S]Met), the carrier-free [³²p] orthophosphoric acid, and the Amplify intensifying reagent were purchased from Amersham Corp., Arlington Heights, II., U.S.A The nitrocellulose membranes were purchased from Bio-Rad (Hercules, Calif., U.S.A.). A mouse monoclonal antibody specific to human TNF (TNF-1) and polyclonal sheep and rabbit anti-human TNF sera were developed in our laboratories. Human IgG, FITC-labeled goat anti-mouse IgG F(ab)′2, non-immune sheep serum, and horseradish peroxidase-conjugated goat anti-rabbit IgG were purchased from BioMaker (Rehovot, Israel).

(b) Cell Culture:

Human acute monocytic leukemia Mono Mac 6 cells (MM6 [Ziegler-Heitbrock et al., 1988]) were obtained from the German Collection of Microorganisms and Cell Cultures. They were grown at a cell density range of 0.3-1×10⁶ cells/ml in RPMI 1640 medium supplemented with 10% fetal calf serum (FCS), 2 mM L-glutamine, 1 mM Na-pyruvate, 1% non-essential amino acids, 9 82 g/ml bovine insulin, 100 U/mI penicillin and 100 g/ml streptomycin. Epithelioid cervical carcinoma HeLa cells (Gey et al., 1952) were obtained from the American Type Culture Collection (Rockville, Md., U.S.A.). The HeLa-M9 cells are a clone of HeLa cells which constitutively express, under control of the SV40 promoter, a TNF mutant cDNA in which the arginine at position +2 and the serine at position +3 were substituted with threonines (the pstA11 construct). These mutations cause an about ten-fold reduction in the cleavage rate of 26 kD TNF (unpublished study). The HeLa and HeLa-M9 cells were grown in RPMI 1640 medium supplemented with 10% FCS, 100 U/ml penicillin, 100 g/ml streptomycin and 50 μg/ml gentamicin.

(c) Indirect Immunofluorescence:

Indirect immunofluorescence analysis was performed as described previously (Pocsik et al., 1994). Briefly, samples of 5×10⁵ cells were incubated for 30 min at 4° C. in the presence of 10 μg/ml mouse monoclonal antibody against human TNF (TNF-1) in phosphate buffered saline (PBS), containing 2 mg/ml BSA, 2 mg/ml human IgG and 0.1% sodium azide, and then with FITC-conjugated goat anti-mouse IgG F(ab)′2, followed by fixation with 1% formaldehyde. Samples of 5,000 cells were analyzed by FACScan (Becton Dickinson, Mountain View, Calif., U.S.A.).

(d) Metabolic Labeling:

Labeling of cells with [³⁵S]Met or [³²P) orthophosphate was performed by incubation in Met-free or phosphate-free medium, supplemented with 5 or 10% FCS, that had been dialyzed against either PBS or 0.9% NaCl, respectively. Unless otherwise indicated, [³⁵S]Met and [³²p] orthophosphate were added to the cells for 2.5 h, at concentrations of 100 μCi/ml and 50 μCi/ml, respectively. Labeling with [³⁵S]Met was performed after a 15 min preincubation in Met-free medium. In the experiments with LPS-stimulated MM6 cells, treatment with LPS was done simultaneously with the metabolic labeling.

(e) Immunoprecipitation and Gel Electrophoresis:

Immunoprecipitation was performed using sheep anti-TNF antiserum or, as a control, non-immune sheep serum, at a dilution of 1:200. To specifically immunoprecipitate cell surface TNF, the antisera, diluted in PBS containing 0.1% BSA and 0.05% sodium azide, were added to the cells prior to their lysis. The cells were incubated for 30 min with the antisera and then rinsed with ice cold PBS. To also immunoprecipitate intracellular TNF molecules, the antisera were directly added to the cell lysate, for a period of 2 h. Cell lysis was performed by incubating the cells for 30 min at a cell concentration of 1×10⁷ cells/ml in a lysis buffer comprised of 50 mM Tris-HCl, pH 7.4, 0.1M NaCl, 1% Triton X-100, 5 mM EDTA, 0.02% sodium azide, 0.1 mM PMSF, and 2 μg/ml leupeptin, and followed by centrifugation at 12,000×g for 15 min to sediment insoluble material. In the ³²P labeling experiments, the lysis buffer was supplemented with 100 μM Na-orthovanadate, 1 mM EGTA and 50 mM NaF. Precipitation of the antibodies was done using protein G-Sepharose beads. All immunoprecipitation steps were performed at 4° C. The immunoprecipitated proteins were analyzed by SDS-PAGE under reducing conditions (12% acrylamide). Gels used for the analysis of [³⁵S] labeled proteins were treated with the Amplify intensifying reagent.

(f) Western Analysis:

Following SDS-PAGE analysis, proteins were Western-blotted to nitrocellulose sheets (Schleicher & Schuell, Dassel, Germany). The blots were probed either with rabbit anti-TNF antibody, followed by incubation with horseradish peroxidase-conjugated goat anti-rabbit IgG and developed with diaminobenzidine-tetrahydrochloride, or with [¹²⁵I] rabbit anti-TNF antibody labeled with the Iodogen reagent ([Aggarwal and Essalu, 1987], 1×10⁷ CPM/blot).

(g) Phosphoamino Acid Analysis:

To identify the phosphorylated amino acid residue(s) in TNF, [³²p] labeled TNF was isolated from extracts of HeLa-M9 cells that had been labeled by incubation for 5 h in growth medium containing 500 μCi [³²p] orthophosphate/ml. The labeled amino acids in the protein were identified as described by Boyle et al., 1991. Briefly, following immunoprecipitation and SDS-PAGE analysis, the protein was blotted onto Immobilon PVDF membrane (Millipore, Bedford, Mass., U.S.A.). The 26 kDa TNF band, identified by autoradiography, was excised from the membrane and hydrolyzed in 6N HCl for 1 h at 110° C. The resulting hydrolysate, to which 0.3 μg of each non-labeled phosphoamino acid marker was added, was fractionated by high voltage two-dimensional thin layer chromatography. The position of the labeled residues, detected by 4 day exposure for autoradiography, was compared with those of the non-labeled residues, as determined by ninhydrin staining.

### EXAMPLE 1

### Cell Surface TNF in HeLa-M9 Cells and in LPS-Treated MM6 Cells

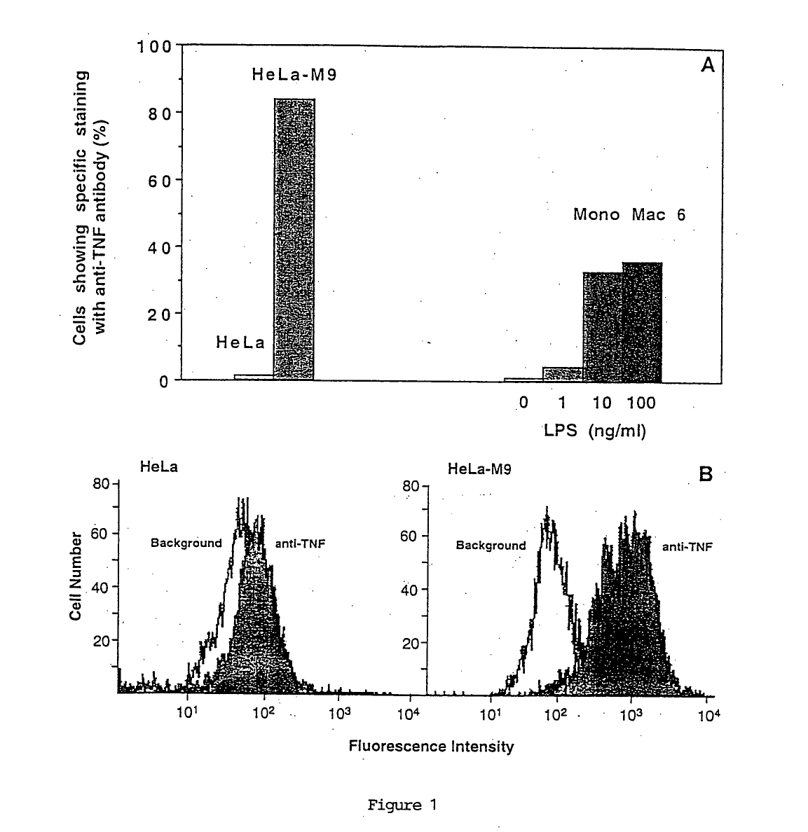

Two cellular systems were employed in this study for characterizing the 26 kDa TNF precursor: (i) HeLa cells that constitutively express transfected cDNA coding for mutated TNF exhibiting reduced processing rates and (ii) cells of the human monocytic leukemia line Mono Mac 6 (MM6 ), which produce the TNF precursor upon LPS stimulation (Pardines-Figueres and Raetz, 1992). As determined by FACS analysis using monoclonal anti-TNF antibody, both the TNF-transfected HeLa cells (HeLa-M9 cells) and the MM6 cells express TNF on their surface (FIG. 1). In the MM6 cells, treatment with LPS resulted in enhanced cell-surface TNF expression, showing maximal effect at 10-100 ng of LPS per ml. These results are set forth in FIGS. 1A and B: FIG. 1A shows a graphic representation of the flow cytometric analysis data of cell surface TNF expression in HeLa cells, HeLa-M9 cells, and MM6 cells treated for 2 h with LPS at concentrations ranging from 0-100 ng/ml (i.e. concentrations of 0, 1, 10 and 100 ng/ml LPS). The amount of cell surface TNF was determined by quantitation (using flow cytometry) of the percentage (%) of cells showing specific staining with the anti-TNF antibody. FIG. 1B shows the FACS profiles (cell number vs. fluorescence intensity) of HeLa and HeLa-M9 cells stained with the anti-TNF antibody (filled curves, denoted “anti-TNF”), and as a control, there is also shown the FACS profiles of cells stained in the absence of anti-TNF antibody (empty curves, denoted “Background”). Furthermore, the signal observed in the FACS analysis was not affected by treating the cells with high salt concentration following fixation, indicating that the TNF molecules are integral to the cell membrane and not soluble molecules adsorbed to the cells (data not shown).

### EXAMPLE 2

### SDS-PAGE and Western Blotting Analysis of TNF Expressed in HeLa-M9 and LPS-treated MM6 Cells

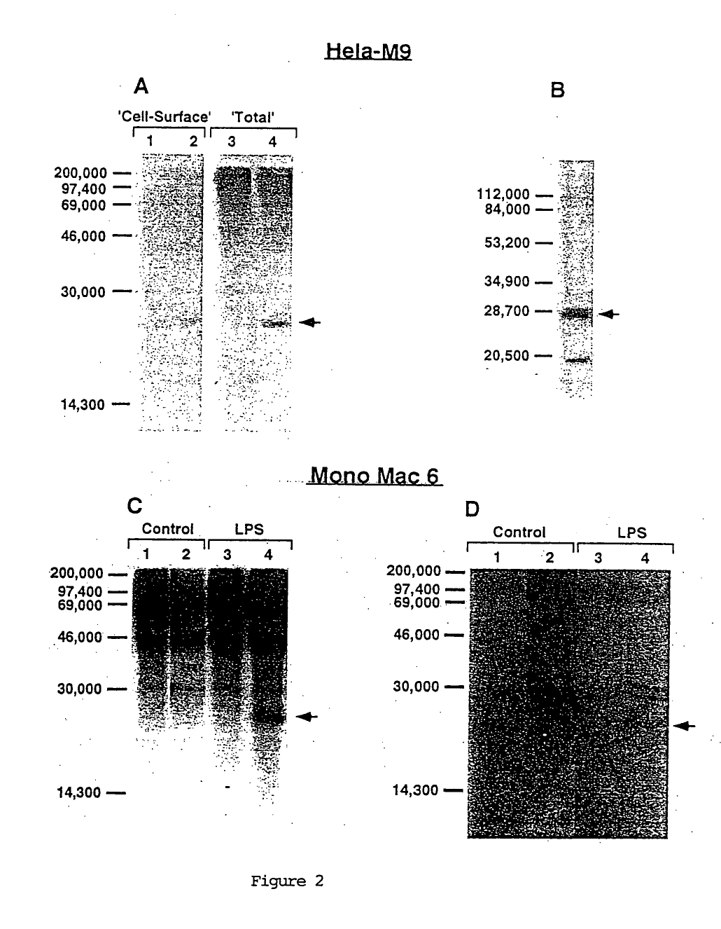

Immunoprecipitation studies revealed that the cell-surface protein recognized by anti-TNF antibodies is the 26 kDa TNF precursor. Two methods of immunoprecipitation were employed: (i) anti-TNF antibodies were incubated with TNF-producing cells prior to cell lysis, thus allowing the antibodies to interact only with cell-surface TNF molecules; and (ii) anti-TNF antibodies were added to the cells following lysis, permitting them to also interact with intracellular TNF molecules. FIGS. 2A-D show the SDS-PAGE and Western blotting analysis results obtained from the above immunoprecipitation procedures: FIGS. 2A and B show the results with respect to the TNF expression in HeLa-M9 cells, and FIGS. 2C and D show the results with respect to the TNF expression in MM6 cells.

More specifically, FIG. 2A shows a reproduction of an autoradiogram of an SDS-PAGE gel on which were separated the following samples: In lanes 2 and 4 are proteins that were immunoprecipitated with anti-TNF antibody from lysates of HeLa-M9 cells that had been metabolically labeled with [³⁵S]Met; and in lanes 1 and 3 are proteins that were immunoprecipitated with control serum from lysates of HeLa-M9 cells metabolically labeled with [³⁵S]Met. Immunoprecipitation was performed by applying the antibodies either before cell lysis, followed by removal of non-bound antibodies, to specifically detect the cell surface TNF (lanes 1 and 2, denoted “cell surface”), or, after lysis to also detect intracellular TNF molecules (lanes 3 and 4, denoted “total”).

FIG. 2B shows a reproduction of an autoradiogram of a Western blot obtained from the Western blotting analysis of proteins in the lysate of HeLa-M9 cells that react with the anti-TNF antibody.

FIG. 2C shows a reproduction of an autoradiogram of an SDS-PAGE gel on which were separated the following samples: In lanes 2 and 4 are proteins that were immunoprecipitated with anti-TNF antibody from lysates of MM6 cells metabolically labeled with [³⁵S]Met; and in lanes 1 and 3 are proteins that were immunoprecipitated with control serum from lysates of MM6 cells metabolically labeled with [³⁵S]Met. The immunoprecipitations were performed in lysates from cells treated with 100 ng/ml LPS for 2 h (lanes 3 and 4, denoted “LPS”) or untreated cells (lanes 1 and 2, denoted “control”).

FIG. 2D shows a reproduction of a Western blot obtained from the Western blotting analysis of the binding of the anti-TNF antibody (lanes 2 and 4) or a control antibody (lanes 1 and 3) to the proteins in lysates of MM6 cells that had been treated with 100 ng/ml LPS for 2 h (lanes 3 and 4, denoted “LPS”) or untreated cells (lanes 1 and 2, denoted “control”).

In all of FIGS. 2A-D there is indicated (on the left hand side) the positions (migration pattern) of the standard molecular weight (M.W.) marker proteins (the M.W. of each marker being shown in daltons). It should also be noted that the development of the Western blots (FIGS. 2B and D) was performed using radiolabeled anti-TNF antibody (FIG. 2D), as set forth above in the “General Procedures and Materials”. Furthermore, the protein samples applied for analysis in each of the above procedures were obtained from the following number of cells: In FIG. 2A, lanes 1 and 2—1.8×10⁶ cells, and lanes 3 and 4—0.6×10⁶ cells; in FIG. 2B—1.8×10⁶ cells; in FIG. 2C (all lanes)—2×10⁶ cells; and in FIG. 2D (all lanes)—1×10⁶ cells.

Thus, as is apparent from FIGS. 2A-D, following both immunoprecipitation methods, there was observed specific recognition of the 26 kDa protein in [³⁵S]Met labeled HeLa-M9 cells. Much greater amounts of the protein were immunoprecipitated if antibodies were added after cell lysis than before lysis, suggesting that most of the 26 kDa TNF molecules occur within the HeLa-M9 cells (compare lanes 3, 4 to 1, 2 in FIG. 2A). Western blotting analysis revealed that, in addition to the 26 kD TNF molecules, lysates of HeLa-M9 cells contain some 17 kDa TNF molecules (FIG. 2B). These molecules could not be detected by labeling with [³⁵S]Met since the 17 kDa TNF does not contain methionine. We also observed the 26 kDa TNF in lysates of LPS-stimulated MM6 cells (FIGS. 2C and D), although in much lower amounts. TNF molecules could be detected when the antibodies were added to the MM6 cells after lysis but not before lysis (data not shown). TNF was not detectable in non-stimulated MM6 cells (lanes 1 and 3 in FIGS. 2C and D), or in HeLa cells which had not been transfected with the TNF cDNA (not shown).

### EXAMPLE 3

### Phosphorylation of the 26 kDa TNF Molecules in HeLa-M9 and LPS-Treated MM6 Cells

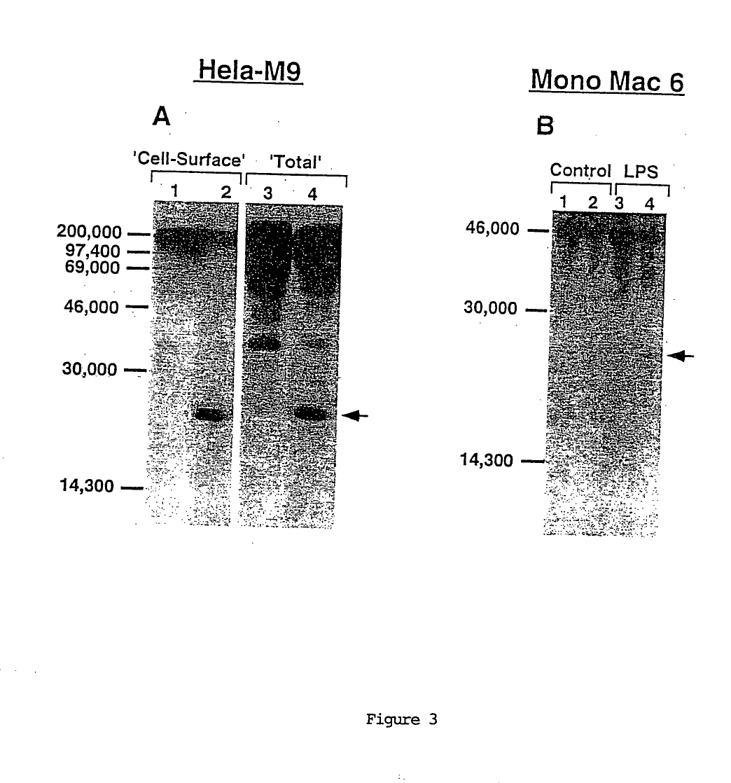

In both the HeLa-M9 cells and LPS activated MM6 cells, growth in the presence of [³²p] resulted in the incorporation of the [³²P]) label in the 26 kDa TNF precursor molecules. These results are shown in FIGS. 3A and B which are reproductions of autoradiograms of SDS-PAGE gels on which were separated [³²P]-labeled proteins that were immunoprecipitated with anti-TNF antibodies (lanes 2 and 4) or control antibodies (lanes 1 and 3) from the lysates of cells that had been metabolically labeled with [³²P] orthophosphate. FIG. 3A shows the proteins immunoprecipitated from the lysates of HeLa-M9 cells, in which the immunoprecipitation was performed by adding the antibodies (anti-TNF or control antibodies—see above) either before cell lysis, followed by the removal of non-bound antibodies, to specifically detect cell-surface TNF molecules (lanes 1 and 2, denoted “cell surface”), or after lysis to detect the intracellular and cell-surface TNF molecules (lanes 3 and 4, denoted “total”). FIG. 3B shows the proteins immunoprecipitated from lysates of MM6 cells that had been treated with 100 ng/ml LPS for 2 h (lanes 3 and 4, denoted “LPS”) or untreated cells (lanes 1 and 2, denoted “control”). It should be noted that the protein samples applied for analysis were from the following numbers of cells: In the analysis depicted in FIG. 3A: lanes 1 and 2—1.8×10⁶ cells; and lanes 3 and 4—0.6×10⁶ cells. In the analysis depicted in FIG. 3B: all lanes—2×10⁶ cells.

Thus, it is apparent from the results shown in FIGS. 3A and B, that as in the [³⁵S]Met labeling experiments (see FIGS. 2A-D), the amount of cell surface [³²P] radiolabeled TNF in the MM6 cells was too low to be detected, though we did find radiolabeled TNF in the whole cell lysate (FIG. 3B, lane 4, denoted by the arrow). Yet, we could isolate [³²P] labeled TNF molecules in the HeLa-M9 cells using both ways of immunoprecipitation (FIG. 3A, lanes 2 and 4), indicating that the cell surface TNF molecules are phosphorylated. No label could be discerned in the 17 kDa form of TNF (compare FIG. 3A lane 4, to FIG. 2B).

### EXAMPLE 4

### Phosphoamino Acid Analysis of the 26 kDa TNF Molecule

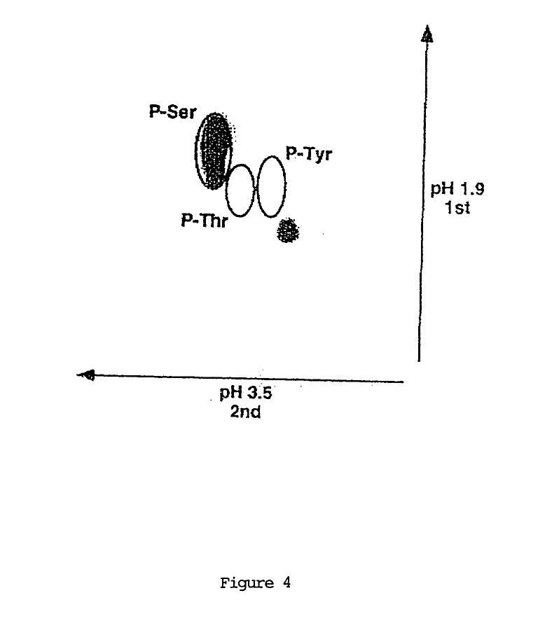

Phosphoamino acid analysis showed that the label in the 26 kDa TNF molecules expressed in HeLa-M9 cells is bound to serine residues. These results are shown in FIG. 4 which is a reproduction of a ninhydrin-stained two-dimensional thin layer electrophoretic analysis of the phosphoamino acids in TNF molecules immunoprecipitated (using anti-TNF antibodies) from the lysates of HeLa-M9 cells metabolically labeled with [³²P] orthophosphate. In this analysis the TNF was immunoprecipitated from the labeled cells, hydrolyzed and subjected to two-dimensional thin layer electrophoresis. FIG. 4 also shows the positions of the unlabeled (cold) internal phosphoamino acid standards as determined by ninhydrin staining. From the positions of these internal standards it was determined that the label in 26 kDa TNF molecules is bound to serine residues.

### EXAMPLE 5

### Cloning and Isolation of Proteins which Bind to the Intracellular Domain of the 26 kDa TNF

To isolate proteins interacting with the intracellular domain of the 26 kDa TNF, the yeast two-hybrid system (Fields and Song, 1989) may be used as described in co-pending Israel patent application Nos. 109632, 112002 and 112742. Briefly, this two-hybrid system is a yeast-based genetic assay to detect specific protein-protein interactions in vivo by restoration of a eukaryotic transcriptional activator such as GAL4 that has two separate domains, a DNA binding and an activation domain, which domains when expressed and bound together to form a restored GAL4 protein, is capable of binding to an upstream activating sequence which in turn activates a promoter that controls the expression of a reporter gene, such as lacZ or HIS3, the expression of which is readily observed in the cultured cells. In this system the genes for the candidate interacting proteins are cloned into separate expression vectors. In one expression vector the sequence of the one candidate protein is cloned in phase with the sequence of the GAL4 DNA-binding domain to generate a hybrid protein with the GAL4 DNA-binding domain, and in the other vector the sequence of the second candidate protein is cloned in phase with the sequence of the GAL4 activation domain to generate a hybrid protein with the GAL4-activation domain. The two hybrid vectors are then co-transformed into a yeast host strain having a lacZ or HIS3 reporter gene under the control of upstream GAL4 binding sites. Only those transformed host cells (cotransformants) in which the two hybrid proteins are expressed and are capable of interacting with each other, will be capable of expression of the reporter gene. In the case of the lacZ reporter gene, host cells expressing this gene will become blue in color when X-gal is added to the cultures. Hence, blue colonies are indicative of the fact that the two cloned candidate proteins are capable of interacting with each other.

Using this two-hybrid system, the intracellular domain of the 26 kDa TNF or portions thereof may be cloned into the vector pGBT9 (carrying the GAL4 DNA-binding sequence, provided by CLONTECH, USA, see below), to create fusion proteins with the GAL4 DNA-binding domain. As the sequence of the intracellular domain of the 26 kDa TNF is known, the DNA sequence encoding the entire domain or portions thereof may be readily isolated and cloned, by standard procedures into the pGBT9 vector utilizing the vector's multiple cloning site region (MCS).

The above hybrid (chimeric) pGBT9 vectors can then be cotransfected together with a cDNA or genomic DNA library from human or other mammalian origin, e.g. a cDNA library from human HeLa cells cloned into the pGAD GH vector, bearing the GAL4 activating domain, into the HF7c yeast host strain (all the above-noted vectors, pGBT9 and pGAD GH carrying the HeLa cell cDNA library, and the yeast strain are purchasable from Clontech Laboratories, Inc., USA, as a part of MATCHMAKER Two-Hybrid System, #PT1265-1). The co-transfected yeasts are then selected for their ability to grow in medium lacking Histidine (His⁻ medium), growing colonies being indicative of positive transformants. The selected yeast clones were then tested for their ability to express the lacZ gene, i.e. for their LAC Z activity, and this by adding X-gal to the culture medium, which is catabolized to form a blue colored product by β-galactosidase, the enzyme encoded by the lacZ gene. Thus, blue colonies are indicative of an active lacZ gene. For activity of the lacZ gene, it is necessary that the GAL4 transcription activator be present in an active form in the transformed clones, namely that the GAL4 DNA-binding domain encoded by one of the above hybrid vectors be combined properly with the GAL4 activation domain encoded by the other hybrid vector. Such a combination is only possible if the two proteins fused to each of the GAL4 domains are capable of stably interacting (binding) to each other. Thus, the His⁺ and blue (LAC Z⁺) colonies that are isolated are colonies which have been cotransfected with a vector encoding a 26 kDa TNF intracellular domain or portion thereof and a vector encoding a protein product of, for example, human HeLa cell origin that is capable of binding stably to the 26 kDa TNF intracellular domain or portion thereof.

The plasmid DNA from the above His⁺, LAC Z⁺ yeast colonies can then be isolated and electroporated into E. coli strainHB101 by standard procedures followed by selection of Leu⁺ and Ampicillin resistant transformants, these transformants being the ones carrying the hybrid pGAD GH vector which has both the Amp^{R} and Leu² coding sequences. Such transformants therefore are clones carrying the sequences encoding newly identified proteins or peptides capable of binding to the intracellular domain of the 26 kDa TNF or a portion thereof. Plasmid DNA is then isolated from these transformed E. coli and retested by
(a) retransforming them with the original 26 kDa TNF intracellular domain-containing hybrid plasmids into yeast strain HF7 as set forth hereinabove. As controls, vectors carrying irrelevant protein encoding sequences, e.g. pACT-larnin or pGBT9 alone can be used for cotransformation with the 26 kDa TNF intracellular domain-binding protein or peptide encoding plasmids. The cotransformed yeasts can then be tested for growth on His⁻ medium alone, or with different levels of 3-aminotriazole; and
(b) retransforming the plasmid DNA and original 26 kDa TNF intracellular domain hybrid plasmids and control plasmids described in (a) into yeast host cells of strain SFY526 and determining the LAC Z⁺ activity (effectivity of β-gal formation, i.e. blue color formation). It should be noted that the above noted β-galactosidase (β-gal) expression tests can also be done by a standard filter assay.

### EXAMPLE 6

### Assessment of the Involvement of Sequence Features Characteristic of the Intracellular Domain of the 26 kDa TNF in the Binding of the Cloned Proteins

The cDNA encoding the protein that contains the intracellular domain of the 26 kDa TNF will be mutated at the various amino acids that constitute this domain. For example, one or more of the serine residues which are phosphorylated in the intracellular domain of the 26 kDa TNF can be replaced by an amino acid residue which is normally not a substrate for phosphorylation, e.g. alanine. Such mutation can be performed, for example, by the Kunkel oligonucleotide-directed mutagenesis procedure. The mutated, as well as the wild-type proteins, can be produced in bacteria as fusions with Glutathione S-transferase (GST). The binding of the cloned 26 kDa TNF intracellular domain binding-protein in vitro to the GST fusion with the mutated 26 kDa TNF intracellular domain will be compared to its binding to the GST-wild type 26 kDa TNF intracellular domain fusion product. Abolition of the binding by the mutation will indicate that the cloned 26 kDa TNF intracellular domain binding-protein indeed recognizes sequence features that are inclusive of the serine residue that was replaced, and hence indicate that the cloned 26 kDa TNF intracellular domain-binding protein is in some way related to the phosphorylation of the serine residue in the intracellular domain of the 26 kDa TNF, e.g. it may be a kinase enzyme, or it may be some other protein, factor, enzyme, etc. which recognizes a phosphorylated or non-phosphorylated serine and by binding to either thereof it modulates the activity of the 26 kDa TNF. A similar approach will be taken to assess the involvement of the other sequence features characteristic of the intracellular domain of the 26 kDa TNF in the function of other reagents that interact with this domain, namely, antibodies, peptides or organic compounds (See Example 7).

### EXAMPLE 7

### Design of Drugs that Affect the 26 kDa TNF by Virtue of Their Ability to Interact with the Intracellular Domain of the 26 kDa TNF

Organic molecules or peptides that interact with the intracellular domain of the 26 kDa TNF will be defined either by screening or by design. Further changes will then be introduced into this molecule to increase the effectivity of its interaction with the intracellular domain of the 26 kDa TNF and the ability of the designed compound to affect (enhance or interfere with) the function of the 26 kDa TNF. Once creating such a molecule and defining the sequence feature of the intracellular domain of the 26 kDa TNF which it recognizes (see Example 6) as well as the conformational features of the intracellular domain of the 26 kDa TNF involved in this recognition (by NMR, X-ray crystallography, etc.), this knowledge can be applied as a starting point for designing drugs that will affect other proteins containing an intracellular domain that shares at least some homology with the 26 kDa intracellular domain, e.g. other members of the TNF/NGF family. To do so, one should introduce to the designed peptide or organic molecule, besides structural features that allow recognition of those structural features that are specific to the intracellular domain of the 26 kDa TNF, also structural features that will dictate specific recognition of the specific other 26 kDa TNF-like intracellular domain-containing protein.

### EXAMPLE 8

### Analysis of the Biological Activity of the 26 kDa TNF Intracellular Domain-Binding Proteins, Peptides, Antibodies or Organic Molecules

Once the 26 kDa TNF intracellular domain-binding proteins or peptides have been isolated, e.g. by the procedure of Example 5, they can be tested for their biological activity. In co-pending applications IL 109632, 111125, 112002 and 112742 there is described one such procedure which assays the effect of various intracellular domain-binding proteins on the cytotoxic effects mediated by the intracellular domains of the p55 TNF-R, FAS-R and MORT1 (HF1) proteins.

Thus, using similar procedures it is possible to determine, firstly, the ability of the 26 kDa TNF intracellular domain-binding proteins or peptides to associate in vitro with the 26 kDa TNF intracellular domain; and secondly to assess in vivo, using standard cell cytotoxicity assays, whether such 26 kDa TNF intracellular domain-binding proteins or peptides are capable of enhancing or inhibiting the amount, activity, etc. of the TNF produced by the TNF-producing cells.

Likewise, the same tests may also be applied to assay organic compounds (obtained by screening or design, see Example 7); synthetically produced peptides (see Example 7); and antibodies, capable of binding to the intracellular domain of the 26 kDa TNF.

### REFERENCES

Aggarwal, B. B. and Vilcek, J.: in Tumor Necrosis Factors: Structure, Function, and Mechanism of Action. New York, Marcel Dekker, Inc. 1991.
Pennica, D., Nedwin, G. E., Hayflick, J. S., Seeburg, P. H., Derynck, R., Paladino, M. A., Kohr, W. J., Aggarwal, B. B. and Goeddel, D. V., (1984) Nature (Lond). 312:724-729.
Kriegler, M., Perez, C., DeFay, K., Albert, I. and Lu, S. D. (1988) Cell 53:45-53.
Perez, C., Albert, I., DeFay, K., Zachariades, N., Gooding, L. and Kriegler, M. (1990) Cell 63:251-258.
Jue, D.-M., Sherry, B., Luedke. C., Manogue, K. R. and Cerami, A. (1990) Biochemistry 29:8371-8377.
Decker, T., Lohmann-Matthes, M.-L. and Gifford, G. E. (1987) J. Immunol. 138:957-962.
Peck, R., Brockhaus, M. and Frey, J. R. (1989) Cell Immunol. 122:1-10.
Duerksen-Hughes, P. J., Day, D. B., Laster, S. M., Zachariades, N. A., Aquino, L. and Gooding, L. R. (1992) J. Immunol. 149:2114-2122.
Nii, A. N., Sone, S., Orino, E. and Ogura, T. (1993) J. Leu. Biol. 53:29-36.
Ratner, A. and Clark, W. R. (1993) J. Immunol. 150:4303-4314.
Lopez-Cepero, M., Garcia-Sanz, J. A., Herbert, L., Riley, R., Handel, M. E., Podack, E. R. and Lopez, D. M. (1994) J. Immunol. 152:3333-3341.
Birkland, T. P., Sypek, J. P. and Wyler, D. J. (1992) J. Leu. Biol. 51:296-299.
Wallach, D. (1986) Interferon 7:89-124.
Van Ostade, X., Tavernier, J. and Fiers, W. (1994) Protein Engineering 7:5-22.
Ziegler-Heitbrock, L., Thiel, E., Futterer, A., Herzog, V., Wirtz, A. and Riethmuller, G. (1988) Eur. J. Cancer 41:456-461.
Gey, G. O., Coffnan, W. D. and Kubicek, M. T. (1952) Cancer Res. 12:264-265.
Pocsik, E., Mihalik, R., Ali-Osman, F. and Aggarwal, B. B. (1994) J. Cell Biochem. 54:453-464.
Aggarwal, B. B. and Essalu, T. E. (1987) J. Biol. Chem. 262:10000-10007.
Boyle, W. J., van der Geer, P. and Hunter, T. (1991) Methods in Enzymol. 201:110-149.
Pardines-Figueres, A. and Raetz, R. H. (1992) J. Biol. Chem. 267:23261-23268.
Liu, M. K., Herrera-Velit, P., Brownsey, R. W. and Reiner, N. E. (1994). J. Immunol. 153:2642-2652.
Han, J., Lee, J.-D., Bibbs, L. and Ulevitch, R. J. (1995) Science 265:808-811.
Kennelly, P. J. and Krebs, E. G. (1991) J. Biol. Chem. 266:15555-15558.
Bosenberg, M. W., Pandiella, A. and Massague, J. (1992) Cell 71:1157-1165.
Crowe, P. D., VanArsdale, T. L., Goodwin, R. G. and Ware, C. F. (1993) J. Immunol. 151:6882-6890.
Brakebusch, C., Nophar, Y., Kemper, O., Engelmann, H. and Wallach. D. (1992) EMBO J. 11:943-950.
Brakebusch, C., Varfolomeev, E. E., Batkin, M. and Wallach, D. (1994) J. Biol. Chem. 269:32488-32496.
Takahashi, T., Tanaka, M., Inazawa, J., Abe, T., Suda, T. and Nagata, D. (1994). Intl. Immunol. 6:1567-1574.
Bazan, J. F. (1993) Current Biology 3:603-606.
Barinaga, M. (1993) Science 2621512-4.
Cantor, G. H. et al. (1993) Proc. Natl. Acad. Sci. USA 90:10932-6.
Chen, C. J. et al. (1992) Ann N.Y. Acad. Sci. 660:271-3.
Crisell, P. et al., (1993) Nucleic Acids Res. (England) 21 (22):5251-5.
Fields, S. and Song, O. (1989) Nature, 340:245-246.
Joseph, S. and Burke, J. M. (1993) J. Biol. Chem. 268:24515-8.
Koizumi, M. et al. (1993) Biol. Pharm. Bull (Japan) 16 (9):879-83.
Sambrook et al. (1989) Molecular Cloning: A Laboratory Manual, Cold Spring Harbor Laboratory Press, Cold spring Harbor, N.Y.
Shimayama, T. et al., (1993) Nucleic Acids Symp. Ser. 29:177-8
Shore, S. K. et al. (1993) Oncogene 8:3183-8.
Wilks, A. F. et al. (1989) Proc. Natl. Acad. Sci. USA, 86:1603-1607.
Zhao, J. J. and Pick, L. (1993) Nature (England) 365:448-51.

1. A method for identifying and producing a molecule which cause modulation of the phosphorylation of the intracellular domain of the 26 kDa TNF, comprising:
a) screening molecules by screening each molecule to determine if the molecule causes modulation of the phosphorylation of the intracellular domain of the 26 kDa TNF by increasing or decreasing the extent of said phosphorylation; and
b) producing in substantially isolated and purified form any said molecule which is determined to cause modulation.

2. The method according to claim 1, wherein said screening step comprises testing each molecule for binding to the intracellular domain of the 26 kDa TNF and then determining if a molecule found to bind to the intracellular domain of the 26 kDa TNF in said testing step modulates the phosphorylation of the intracellular domain of the 26 kDa TNF.
